(19) United States
(12) Patent Application Publication
(10) Pub. No.: US 2004/0110952 A1
(43) Pub. Date: Jun. 10, 2004

(54) N-4-piperidinyl compounds as ccr5 modulators

(76) Inventors: Jeremy Burrows, Cheshire (GB); John Cumming, Cheshire (GB); Howard Tucker, Cheshire (GB)

Correspondence Address: Janis K Fraser, Fish & Richardson, 225 Franklin Street, Boston, MA, 02110-2804, US

(21) Appl. No.: 10/469,361

(22) PCT Filed: Feb. 27, 2002
(86) PCT No.: PCT/SE02/00351

(30) Foreign Application Priority Data
Mar. 1, 2001 (GB) 0105077.2
Jun. 26, 2001 (GB) 0115579.5
Nov. 13, 2001 (SE) 0103797-7

(51) Int. Cl. C 07D 4 1/02; C07D 211/68

(52) U.S. Cl. 546/193; 546/207; 546/224

(57) ABSTRACT

The invention provides a compound of formula (1): wherein R¹, R², R³, R^{3a}, R⁴, R^{4a}, R⁵, and R⁶ are defined; or a pharmaceutically acceptable salt thereof or a solvate thereof; compositions containing these compounds, processes for preparing them and their use as modulators of chemokine activity (especially CCR5 activity).

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in PCT/SE01/01053, EP-A1-1013276, WO00/08013, WO99/38514 and WO99/04794.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1a and MIP-1b and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I):

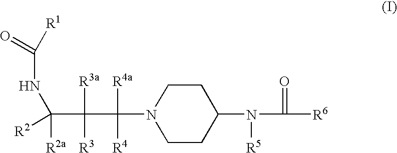

wherein:

R¹ is C₃₋₇ cycloalkyl, C₄₋₇ cycloalkyl fused to a phenyl ring, C₅₋₇ cycloalkenyl, heterocyclyl (itself optionally substituted by oxo or C₁₋₄ alkyl), C₁₋₈ alkyl (substituted by C₃₋₆ cycloalkyl, C₅₋₆ cycloalkenyl, S(O)ₚR⁷ or COR⁸), C₂₋₈ alkenyl or C₂₋₈ alkynyl;

R² is optionally substituted phenyl, optionally substituted heteroaryl or cycloalkyl;

R^{2a}, R⁴ and R^{4a} are, independently, hydrogen or C₁₋₄ alkyl;

R³ and R^{3a} are, independently, hydrogen or C₁₋₄ alkyl or C₁₋₄ alkoxy;

R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;

R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH;

R⁷ and R⁸ are, independently, C₁₋₄ alkyl;

wherein the phenyl and heteroaryl rings of any of the foregoing are independently optionally substituted by halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;

R⁹ and R¹⁰ are, independently, hydrogen of C₁₋₄ alkyl, or together with a nitrogen or oxygen atom, may join to form a 5- or 6-membered ring which is optionally substituted with C₁₋₄ alkyl heteroatom and that heteroatom is nitrogen, then the heterocyclyl ring is not N-linked to the remainder of the structure of formula (I); and provided that when R¹ is cyclobutyl or tetrahydropyran, R² is optionally substituted phenyl, R³ is hydrogen or alkoxy and R⁶ is benzyl (optionally substituted by alkoxy) or pyridinylmethyl, then R^{2a}, R^{3a}, R⁴, R^{4a} and R⁵ are not all hydrogen;

or a pharmaceutically acceptable salt thereof or a solvate thereof.

In one particular aspect the present invention provides a compound of formula (I) wherein R¹, R², R^{2a}, R³, R^{3a}, R⁴, R^{4a}, R⁵ and R⁶ are as defined above; provided that when heterocyclyl contains a one heteroatom and that heteroatom is nitrogen, then the heterocyclyl ring is not N-linked to the remainder of the structure of formula (I); and provided that when R¹ is cycloalkyl or heterocyclyl, R² is optionally substituted phenyl, R³ is hydrogen or alkoxy and R⁶ is benzyl (optionally substituted by alkoxy) or pyridinylmethyl, then R^{2a}, R^{3a}, R⁴, R^{4a} and R⁵ are not all hydrogen; or a pharmaceutically acceptable salt thereof or a solvate thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate or p-toluenesulphonate.

The compounds of the invention may exist as solvates (such as hydrates) and the present invention covers all such solvates.

Alkyl groups and moieties preferably contain, unless otherwise specified, 1-6, especially 1-4, carbon atoms. Alkyl groups and moieties are straight or branched chain and are, for example, methyl, ethyl, n-propyl or iso-propyl.

Alkenyl and alkynyl groups and moieties preferably contain, unless otherwise specified, 2-6, especially 2-4, carbon atoms. Alkenyl includes prop-2-en-1-yl, allyl, but-3-en-1-yl, but-1-en-1-yl, 2-methylallyl, 1-methyl-but-3-en-1-yl, 1-methyl-but-1-en-1-yl, pent-2-en-1-yl and hex-1-en-1-yl. Alkynyl includes propargyl, but-3-yn-1-yl, pent-4yn-1-yl and hex-5-yn-1-yl. Alkenyl and alkynyl groups and moieties are, for example, vinyl, allyl or propargyl.

Cycloalkyl preferably contains, unless otherwise specified, 3-7, especially 3-6, carbon atoms. Cycloalkyl is, for example, cyclopropyl, cyclobutyl or cyclopentyl.

Cycloalkyl fused to a phenyl ring is, for example, benzocyclobuten-1-yl, indan-1-yl or indan-2-yl.

Heterocyclyl is a non-aromatic, mono- or bicyclic 3, 4, 5, 6, 7 or 8 membered ring system comprising at least one heteroatom selected from the group comprising nitrogen, oxygen and sulphur. (For example heterocyclyl is a non-aromatic 3, 4, 5 or 6 membered ring comprising at least one heteroatom selected from the group comprising nitrogen, oxygen and sulphur.) Heterocyclyl includes aziridinyl, azetidinyl, oxetanyl, piperidinyl, 4,5-dihydro-oxazolyl, 4,5-dihydroimidazolyl, morpholinyl, thiomorpholinyl, pyrrolidinyl, piperazinyl, tetrahydrofuryl, tetrahydropyran and quinuclidinyl. (For example heterocyclyl is aziridinyl, azetidinyl, oxetanyl, piperidinyl, 4,5-dihydro-oxazolyl, 4,5-dihydroimidazolyl, morpholinyl, pyrrolidinyl, piperazinyl or tetrahydrofuryl.) Substituted heterocyclyl is, for example, azetidinonyl or N-methyl-piperidinyl.

Heteroaryl is an aromatic 5 or 6 membered ring comprising at least one heteroatom selected from the group comprising nitrogen, oxygen and sulphur. Heteroaryl is, for example, pyrrolyl, imidazolyl, pyrazolyl, 1,2,3-triazolyl, 1,2,4-triazolyl, oxazolyl, isoxazolyl, oxadiazolyl, thiazolyl, isothiazolyl, thiadiazolyl, pyridinyl, pyrimidinyl, pyrazinyl, pyridazinyl, thienyl, furyl, quinolinyl, isoquinolinyl, dihydroisoquinolinyl, quinazolinyl, quinoxalinyl, indolyl, isoindolyl, benzimidazolyl, benzo[b]furyl, benzo[b)thienyl, phthalazinyl, benzthiazolyl or cinnolinyl.

Phenylalkyl is, for example, benzyl, 1-(phenyl)eth-1-yl or 1-(phenyl)eth-2-yl.

Heteroarylalkyl is, for example, pyridinylmethyl, pyrimidinylmethyl or 1-(pyridinyl)eth-2-yl.

The group S(O)₂NR⁹R¹⁰ is, for example, S(O)₂NH₂, S(O)₂NH(C₁₋₄ alkyl), S(O)₂N(C₁₋₄ alkyl)₂, S(O)₂(4-C(O)H-piperazin-1-yl) or S(O)₂(4-C(O)CH₃-piperazin-1-yl).

Phenyl(C₁₋₂ alkyl)NH is, for example, benzylamino. Heteroaryl(C₁₋₂ alkyl)NH is, for example, pyridinylCH₂NH, pyrimidinylCH₂NH or pyridinylCH(CH₃)NH.

In one aspect the present invention provides a compound of formula (I), wherein R¹ is C₃₋₇ cycloalkyl, C₄₋₇ cycloalkyl fused to a phenyl ring, C₅₋₇ cycloalkenyl, heterocyclyl (itself optionally substituted by C₁₋₄ alkyl), C₁₋₈ alkyl (substituted by C₃₋₆ cycloalkyl, C₅₋₆ cycloalkenyl, S(O)ₚR⁷ or COR⁸), C₂₋₈ alkenyl or C₂₋₈ alkynyl; R² is optionally substituted phenyl or optionally substituted heteroaryl; R^{2a}, R⁴ and R^{4a} are, independently, hydrogen or C₁₋₄ alkyl; R³ and R^{3a} are, independently, hydrogen or C₁₋₄ alkyl or C₁₋₄ alkoxy; R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl; R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH; R⁷ and R⁸ are, independently, C₁₋₄ alkyl; wherein the phenyl and heteroaryl rings of any of the foregoing are independently optionally substituted by halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; R⁹ and R¹⁰ are, independently, hydrogen or C₁₋₄ alkyl or together with a nitrogen or oxygen atom, may join to form a 5- or 6-membered ring which is optionally substituted with C₁₋₄ alkyl, C(O)H or C(O)(C₁₋₄ alkyl); m, p and q are, independently, 0, 1 or 2; provided that when heterocyclyl contains a one heteroatom and that heteroatom is nitrogen, then the heterocyclyl ring is not N-linked to the remainder of the structure of formula (I); or a pharmaceutically acceptable salt thereof or a solvate thereof.

In a further aspect the present invention provides a compound of formula (I), wherein R¹ is C₃₋₇ cycloalkyl, C₄₋₇ cycloalkyl fused to a phenyl ring, C₅₋₇ cycloalkenyl, heterocyclyl (itself optionally substituted by C₁₋₄ alkyl), C₁₋₈ alkyl (substituted by C₃₋₆ cycloalkyl, C₅₋₆ cycloalkenyl, S(O)ₚR⁷, COR⁸), C₂₋₈ alkenyl or C₂₋₈ alkynyl; R² is optionally substituted phenyl or optionally substituted heteroaryl; R^{2a}, R⁴ and R^{4a} are, independently, hydrogen or C₁₋₄ alkyl; R³ and R^{3a} are, independently, hydrogen or C₁₋₄ alkyl or C₁₋₄ alkoxy; R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl; R⁶ is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH; R⁷ and R⁸ are, independently, C₁₋₄ alkyl; wherein the phenyl and heteroaryl rings of any of the foregoing are independently optionally substituted by halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl); m, p and q are, independently, 0, 1 or 2; or a pharmaceutically acceptable salt thereof or a solvate thereof.

In another aspect the present invention provides a compound of formula (I), wherein R¹ is C₃₋₇ cycloalkyl, C₅₋₇ cycloalkenyl, heterocyclyl (itself optionally substituted by C₁₋₄ alkyl), C₁₋₈ alkyl (substituted by C₃₋₆ cycloalkyl, C₅₋₆ cycloalkenyl, S(O)ₚR⁷, COR⁸), C₂₋₈ alkenyl or C₂₋₈ alkynyl; R² is optionally substituted phenyl or optionally substituted heteroaryl; R^{2a}, R⁴ and R^{4a} are, independently, hydrogen or C₁₋₄ alkyl, R³ and R^{3a} are, independently, hydrogen or C₁₋₄ alkyl or C₁₋₄ alkoxy; R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl; R6 is phenyl, heteroaryl, phenylNH, heteroarylNH, phenyl(C₁₋₂)alkyl, heteroaryl(C₁₋₂)alkyl, phenyl(C₁₋₂ alkyl)NH or heteroaryl(C₁₋₂ alkyl)NH; R⁷ and R⁸ are, independently, C₁₋₄ alkyl; wherein the phenyl and heteroaryl rings of any of the foregoing are independently optionally substituted by halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃; R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl); m, p and q are, independently, 0, 1 or 2; or a pharmaceutically acceptable salt thereof or a solvate thereof.

In another aspect of the invention R¹ is cyclopropyl, cyclobutyl, cyclopentyl, cyclohexenyl, benzocyclobuten-1-yl, indanyl, 5-, 6- or 8-membered, non-N-linked, heterocyclyl (optionally substituted by oxo or methyl), C₁₋₄ alkyl (singly substituted by C₃₋₆ cycloalkyl, C₅₋₆ cycloalkenyl, S(C₁₋₄ alkyl) or CO(C₁₋₄ alkyl)), C₂₋₆ alkenyl or C₂₋₆ alkynyl.

5-, 6- or 8-Membered heterocyclyl includes piperidinyl, 4,5-dihydro-oxazolyl, 4,5-dihydroimidazolyl, morpholinyl, thiomorpholinyl, pyrrolidinyl, piperazinyl, tetrahydrofuryl, tetrahydropyran or quinuclidinyl; and is, for example, piperidinyl, 4,5-dihydro-oxazolyl, 4,5-dihydroimidazolyl, morpholinyl, thiomorpholinyl, piperazinyl, tetrahydrofuryl, tetrahydropyran or quinuclidinyl.

In yet another aspect R¹ is C₄₋₇ cycloalkyl fused to a phenyl ring (for example benzocyclobuten-1-yl or indanyl) or C₅₋₇ cycloalkenyl (for example cyclohexenyl).

In a further aspect R¹ is, for example, cyclopropyl, cyclobutyl, cyclohexenyl, benzocyclobuten-1-yl, 5-membered heterocyclyl (optionally substituted by methyl), C₁₋₄ alkyl (singly substituted by C₅₋₆ cycloalkenyl, S(C₁₋₄ alkyl) or CO(C₁₋₄ alkyl)), C₂₋₆ alkenyl or C₂₋₆ alkynyl.

In a still further aspect R¹ is, for example, cyclopropyl, cyclohexenyl, benzocyclobuten-1-yl, C₁₋₄ alkyl (singly substituted by C₅₋₆ cycloalkenyl, S(C₁₋₄ alkyl) or CO(C₁₋₄ alkyl)), C₂₋₆ alkenyl or C₂₋₆ alkynyl.

In another aspect R¹ is, for example, cyclopropyl, cyclobutyl, cyclohexenyl, 5-membered heterocyclyl (optionally substituted by methyl), C₁₋₄ alkyl (singly substituted by C₅₋₆ cycloalkenyl, S(C₁₋₄ alkyl) or CO(C₁₋₄ alkyl)), C₂₋₆ alkenyl or C₂₋₆ alkynyl.

In yet another aspect R¹ is C₄₋₇ cycloalkyl fused to a phenyl ring, for example benzocyclobuten-1-yl.

In a further aspect R² is phenyl or heteroaryl, either of which is optionally substituted in the ortho or meta position by halo, C₁₋₄ alkyl, C₁₋₄ alkoxy, S(O)ₙ(C₁₋₄ alkyl), nitro, cyano or CF₃. Halo is especially fluorine or chlorine.

In another aspect R² is cyclohexyl or phenyl or heteroaryl, either of which is optionally substituted in the ortho or meta position by halo, C₁₋₄ alkyl, C₁₋₄ alkoxy, S(O)ₙ(C₁₋₄ alkyl), nitro, cyano or CF₃. Halo is especially fluorine or chlorine.

In yet another aspect R² is cyclohexyl or heteroaryl (which is optionally substituted in the ortho or meta position by halo, C₁₋₄ alkyl, C₁₋₄ alkoxy, S(O)ₙ(C₁₋₄ alkyl), nitro, cyano or CF₃). Halo is especially fluorine or chlorine.

In another aspect R² is optionally substituted phenyl (especially optionally substituted by halogen or CF₃). Halogen is especially fluorine or chlorine. For example R² is 3-fluorophenyl, 3-chlorophenyl, 4-fluorophenyl or 4-CF₃-phenyl.

In a still further aspect R²is optionally substituted phenyl (especially optionally substituted by halo, cyano, methyl, ethyl; methoxy, ethoxy, NH₂, NHCH₃, N(CH₃)₂, CF₃, CHF₂, CH₂F, CH₂CF₃ or OCF₃). Halo is especially fluorine or chlorine. It is preferred that said substitution is on the ortho or meta position of the phenyl ring.

In yet another aspect R⁴ and R^{4a} are hydrogen or methyl.

In a further aspect R⁴ and R^{4a} are hydrogen or methyl, and R^{2a}, R³ and R^{3a} are all hydrogen.

In a yet further aspect R⁴ and R^{4a} are, independently, hydrogen or methyl.

In a still further aspect R⁴ and R^{4a} are, independently, hydrogen or methyl (for example R⁴ is hydrogen and R^{4a} is methyl, or R⁴ and R^{4a} are both hydrogen), and R^{2a}, R³ and R^{3a} are all hydrogen.

In a still further aspect R^{2a}, R³, R^{3a}, R⁴ and R^{4a} are all hydrogen.

In another aspect R^{2a} is hydrogen.

In yet another aspect R³ and R^{3a} are both hydrogen.

In a still further aspect R⁴ in hydrogen or methyl and R^{4a} is hydrogen.

In another aspect R⁵ is hydrogen, methyl or ethyl.

In yet another aspect R⁵ is iso-propyl, C₃₋₄ alkenyl, C₃₋₄ alkynyl, C₃₋₇ cycloalkyl or C₃₋₇ cycloalkyl(C₁₋₄ alkyl). For example R⁵ is allyl, propargyl, cyclopropyl or cyclopropylCH₂.

In a further aspect R⁵ is ethyl, allyl or cyclopropyl.

In still further aspects of the invention R⁵ is ethyl; or R⁵ is allyl or cyclopropyl.

In a still further aspect of the invention R⁶ is preferably optionally substituted benzyl, especially benzyl singly substituted (such as in the 4-position) by S(O)₂(C₁₋₄)alkyl (such as S(O)₂CH₃) or S(O)₂NR⁹R¹⁰ {R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl)} (such as S(O)₂NH₂, S(O)₂NH(CH₃), S(O)₂N(CH₃)₂, S(O)₂(4-C(O)H-piperazin-1-yl) or S(O)₂(4-C(O)CH₃-piperazin-1-yl). The 5- or 6-membered ring is, for example, morpholine, thiomorpholine, piperidine, piperazine or pyrrolidine; but is especially piperazine.

In another aspect of the invention R⁶ is benzyl singly substituted (such as in the 4-position) by S(O)₂(C₁₋₄)alkyl (such as S(O)₂CH₃).

In yet another aspect the present invention provides a compound of formula (Ia):

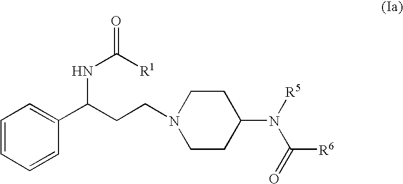

wherein R¹, R⁵ and R⁶ are as defined above.

In yet another aspect the present invention provides a compound of formula (Ib):

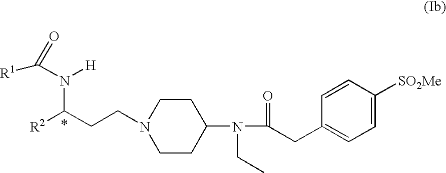

wherein R¹ and R² are as defined above. It is preferred that the compounds of formula (Ib) have S absolute configuration at the asterisked carbon (that is, the carbon labelled ‘*’).

The following compounds illustrate the invention.

TABLE I
Table I comprises compounds of formula (Ia).

| | | | | (Ia) |
|---|---|---|---|---|
| 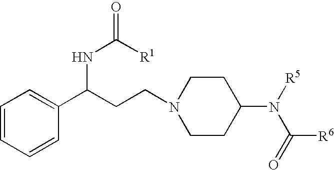 | | | | |
| Compound | | | | LCMS |
| No. | R¹ | R⁵ | R⁶ | (MH+) |
| 1 | CH2-cyclopentyl | Ethyl | CH2Ph-4-SO2Me | 568 |
| 2 | 2-pyrrolidine-1-Me | Ethyl | CH2Ph-4-SO2Me | 569 |
| 3 | 2-tetrahydrofuran | Ethyl | CH2Ph-4-SO2Me | 556 |
| 4 | cyclobutyl | Ethyl | CH2Ph-4-SO2Me | 540 |
| 5 | cyclohexyl | Ethyl | CH2Ph-4-SO2Me | 568 |
| 6 | cyclopentyl | Ethyl | CH2Ph-4-SO2Me | 554 |
| 7 | cyclopropyl | Ethyl | CH3Ph-4-SO2Me | 526 |
| 8 | CH2CH2CCH | Ethyl | CH2Ph-4-SO2Me | 538 |
| 9 | CH2CH2CH═CH2 | Ethyl | CH2Ph-4-SO2Me | 540 |
| 10 | CH2CH2COCH3 | Ethyl | CH2Ph-4-SO2Me | 556 |
| 11 | CH2CH2SCH3 | Ethyl | CH2Ph-4-SO2Me | 560 |
| 12 | CH2CH2CH2CCH | Ethyl | CH2Ph-4-SO2Me | 552 |
| 13 | CH2CH2CH2CH2CCH | Ethyl | CH2Ph-4-SO2Me | 566 |
| 14 | CH2CH2CH2CH2CH═CH2 | Ethyl | CH2Ph-4-SO2Me | 568 |
| 15 | 4-cyclohexene | Ethyl | CH2Ph-4-SO2Me | 566 |
| 16 | C(CH3)═CHCH2CH3 | Ethyl | CH2Ph-4-SO2Me | 554 |
| 17 | CCCH3 | Ethyl | CH2Ph-4-SO2Me | 524 |
| 18 | CH(CH3)CH2CH═CH2 | Ethyl | CH2Ph-4-SO2Me | 554 |
| 19 | CH═CH═CHCH3 | Ethyl | CH2Ph-4-SO2Me | 552 |
| 20 | CH═CHCH2CH3 | Ethyl | CH2Ph-4-SO2Me | 540 |
| 21 | CH═CHCH3 | Ethyl | CH2Ph-4-SO2Me | 526 |
| 22 | CH═C(CH3)2 | Ethyl | CH2Ph-4-SO2Me | 540 |
| 23 | CH2-3-cyclopentene | Ethyl | CH2Ph-4-SO2Me | 566 |
| 24 | CH2CH═CH2 | Ethyl | CH2Ph-4-SO2Me | 526 |
| 25 | CH2CH═CHCH2CH3 | Ethyl | CH2Ph-4-SO2Me | 554 |
| 26 | CH2SCH3 | Ethyl | CH2Ph-4-SO2Me | 546 |
| 27 | cyclobutyl | Allyl | CH2Ph-4-SO2Me | 552 |
| 28 | cyclobutyl | cyclopropyl | CH2Ph-4-SO2Me | 552 |
| 29 | benzocyclobuten-1-yl | Ethyl | CH2Ph-4-SO2Me | 588 |
| 30 | benzocyclobuten-1-yl | Allyl | CH2Ph-4-SO2Me | |
| 31 | benzocyclobuten-1-yl | cyclopropyl | CH2Ph-4-SO2Me | |
| 32 | 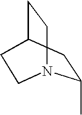 | Ethyl | CH2Ph-4-SO2Me | |
| | 2-quinuclidinyl | | | |
| 33 | 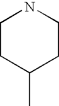 | Ethyl | CH2Ph-4-SO2Me | |
| | 4-piperidinyl | | | |
| 34 | 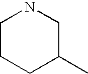 | Ethyl | CH2Ph-4-SO2Me | |
| | 3-piperidinyl | | | |
| 35 | 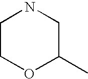 | Ethyl | CH2Ph-4-SO2Me | |
| | 2-morpholinyl | | | |
| 36 | 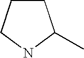 | Ethyl | CH2Ph-4-SO2Me | |
| | 2-pyrrolidinyl | | | |
| 37 | 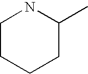 | Ethyl | CH2Ph-4-SO2Me | |
| | 2-piperidinyl | | | |
| 38 | 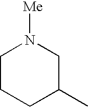 | Ethyl | CH2Ph-4-SO2Me | |
| | 3-(N-methyl)piperidinyl | | | |
| 39 | 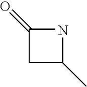 | Ethyl | CH2Ph-4-SO2Me | |
| | 4-azetidin-2-onyl | | | |
| 40 | 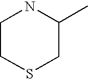 | Ethyl | CH2Ph-4-SO2Me | |
| | 3-thiomorpholinyl | | | |

TABLE II
Table II comprises compounds of formula (Ib).

| | | | | (Ib) |
|---|---|---|---|---|
| 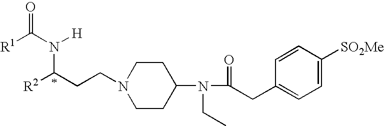 | | | | |
| Com- | | | | |
| pound | | | | LCMS |
| No. | R¹ | R² | Comment | (MH+) |
| 1 | Benzocyclobuten-1-yl | Phenyl | S isomer at * | 588 |
| 2 | Indan-2-yl | Phenyl | S isomer at * | 602 |
| 3 | Tetrahydropyran-4-yl | Phenyl | S isomer at * | 570 |
| 4 | Benzocyclobuten-1-yl | Cyclohexyl | | 594 |
| 5 | Benzocyclobuten-1-yl | 4-Chlorophenyl | | 622 |

The compounds of formulae (I), (Ia) and (Ib) can be prepared as shown in Schemes I below.

Specifically, a compound of formula (I), (Ia) or (Ib) can be prepared by treating a compound of formula (II):

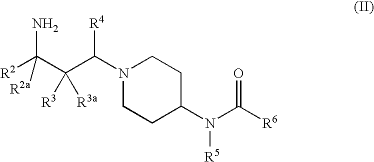

with: an acid chloride of formula R¹C(O)Cl, in the presence of a base (such as potassium carbonate) and in a suitable solvent (such as a chlorinated hydrocarbon, for example dichloromethane); or an acid of formula R¹CO₂H in the presence of a suitable coupling agent (such as O-(7-Azabenzotriazol-1-yl)-N,N,N′,N′-tetramethyluronium hexafluorophosphate [HATU] or bromo-tris-pyrrolidino-phosphonium hexafluorophosphate [PyBrop]) in the presence of a suitable base (such as a tertiary amine, for example diisopropylethylamine) in a suitable solvent (such as N-methylpyrrolidinone).

A compound of formula (II) can be prepared by treating a compound of formula (III):

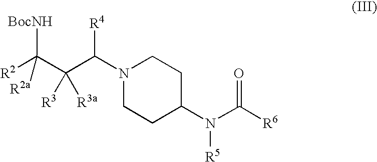

with trifluoroacetic acid or hydrochloric acid in the presence of methanol, and then basifying to release the free amine form of formula (II).

A compound of formula (III) can be prepared by reductively aminating a compound of formula (IV):

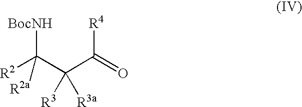

with a compound of formula (V):

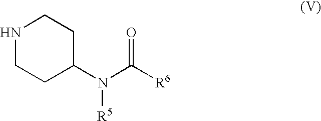

in the presence of a suitable solvent (such as an aliphatic alcohol such as methanol), a suitable organic acid (such as an aliphatic acid, for example acetic acid) and a suitable reducing agent (such as sodium triacetoxyborohydride or sodium cyanoborohydride).

A compound of formula (II) wherein R^{2a} is hydrogen can be prepared by reductive amination of a compound of formula (VI):

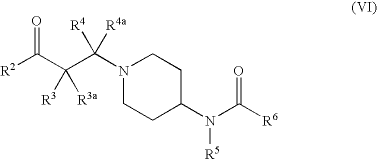

for example by reacting a compound of formula (VI) with hydroxyl amine and hydrogenating the product so formed with hydrogen in the presence of a suitable metal catalyst (such as palladium or platinum catalyst, for example palladium on charcoal).

A compound of formula (VI), wherein R^{4a} is hydrogen, can be prepared by reacting a compound of formula (V) with:

an alkyl halide of formula R²C(O)CR³R^{3a}CHR⁴X (wherein X is halogen, such as chloro, bromo or iodo) in the presence of a suitable base (such as potassium carbonate) and a suitable solvent (such as acetone); or,

compounds of formula R²C(O)CHR³R^{3a} and R⁴CHO in the presence of a suitable acid (such as acetic acid.

A compound of formula (VI), wherein R^{3a} is hydrogen, can be prepared by reacting a compound of formula (V) with an alkene of formula R²C(O)CR³═CR⁴R^{4a} in a suitable solvent (such as an aliphatic alcohol, for example ethanol) at a temperature in the range −10 to 100° C.

The starting materials for these processes are commercially available, can be prepared by literature methods or can be prepared by adapting literature methods. In a further aspect the invention provides processes for preparing the compounds of formulae (I), (Ia) and (Ib). Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (especially CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)). Examples of these conditions are:

(1) (the respiratory tract) obstructive diseases of airways including: chronic obstructive pulmonary disease (COPD) (such as irreversible COPD); pulmonary fibrosis; asthma (such as bronchial, allergic, intrinsic, extrinsic or dust asthma, particularly chronic or inveterate asthma (for example late asthma or airways hyper-responsiveness)); bronchitis (such as eosinophilic bronchitis); acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease; and/or

(6) (other tissues or diseases) Alzheimer's disease, multiple sclerosis, atherosclerosis, inhibiting the entry of viruses into target cells, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura, disorders of the menstrual cycle, glomerulonephritis or cerebral malaria.

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia) or (Ib), or a pharmaceutically acceptable salt thereof or a solvate thereof, for use in a method of treatment of a warm blooded animal (such as man) by therapy (including prophylaxis).

According to a further feature of the present invention there is provided a method for modulating chemokine receptor activity (especially CCR5 receptor activity) in a warm blooded animal, such as man, in need of such treatment, which comprises administering to said animal an effective amount of a compound of the present invention, or a pharmaceutically acceptable salt thereof or a solvate thereof.

The present invention further provides a method of treating a chemokine mediated disease state (especially a CCR5 mediated disease state, such as rheumatoid arthritis) in a warm blooded animal (such as man) suffering from, or at risk of, said disease, which comprises administering to an animal in need of such treatment a therapeutically effective amount of a compound of formula (I), (Ia) or (Ib), or a pharmaceutically acceptable salt thereof or solvate thereof.

The invention also provides a compound of the formula (I), (Ia) or (Ib), or a pharmaceutically acceptable salt thereof or a solvate thereof, for use in therapy (including prophylaxis); for example in the treatment of a chemokine mediated disease state (especially a CCR5 mediated disease state) in a warm blooded animal, such as man, such as in the treatment of rheumatoid arthritis.

The invention also provides a compound of the formula (I), (Ia) or (Ib), or a pharmaceutically acceptable salt thereof or a solvate thereof, for use as a medicament, especially a medicament for the treatment of rheumatoid arthritis.

In another aspect the present invention provides the use of a compound of the formula (I), (Ia) or (Ib), or a pharmaceutically acceptable salt thereof or a solvate thereof, in the manufacture of a medicament for use in therapy (for example in modulating chemokine receptor activity (especially CCR5 receptor activity (especially in the treatment of rheumatoid arthritis)) in a warm blooded animal, such as man).

The invention further provides the use of a compound of formula (I), (Ia) or (Ib), or a pharmaceutically acceptable salt thereof, in the manufacture of a medicament for use in the treatment of:

(1) (the respiratory tract) obstructive diseases of airways including: chronic obstructive pulmonary disease (COPD) (such as irreversible COPD); asthma (such as bronchial, allergic, intrinsic, extrinsic or dust asthma, particularly chronic or inveterate asthma (for example late asthma or airways hyper-responsiveness)); bronchitis (such as eosinophilic bronchitis); acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease; and/or

(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura or disorders of the menstrual cycle;

in a warm blooded animal, such as man.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia) or (Ib), or a pharmaceutically acceptable salt thereof or a solvate thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the 5 compound of formula (I), (Ia) or (Ib), or a pharmaceutically acceptable salt thereof or a solvent thereof (hereafter Compound X), for therapeutic or prophylactic use in humans:

| | mg/tablet |
|---|---|
| (a) | |
| Tablet I | |
| Compound X | 100 |
| Lactose Ph. Eur. | 179 |
| Croscarmellose sodium | 12.0 |
| Polyvinylpyrrolidone | 6 |
| Magnesium stearate | 3.0 |
| (b) | |
| Tablet II | |
| Compound X | 50 |
| Lactose Ph. Eur. | 229 |
| Croscarmellose sodium | 12.0 |
| Polyvinylpyrrolidone | 6 |
| Magnesium stearate | 3.0 |
| (c) | |
| Tablet III | |
| Compound X | 1.0 |
| Lactose Ph. Eur. | 92 |
| Croscarmellose sodium | 4.0 |
| Polyvinylpyrrolidone | 2.0 |
| Magnesium stearate | 1.0 |
| (d) | |
| Capsule | mg/capsule |
| Compound X | 10 |
| Lactose Ph. Eur. | 389 |
| Croscarmellose sodium | 100 |
| Magnesium stearate | 1.0 |
| (e) | |
| Injection I | (50 mg/ml) |
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “MegaBond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.

(iv) in general, the course of reactions was followed by TLC and reaction times are given for illustration only;

(v) yields, when given, are for illustration only and are not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion −(M+H)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion −(M+H)⁺ and

(xi) the following abbreviations are used:

| | |
|---|---|
| DMSO | dimethyl sulphoxide; |
| DMF | N-dimethylformamide; |
| DCM | dichloromethane; |
| NMP | N-methylpyrrolidinone; |
| HATU | O-(7-Azabenzotriazol-1-yl)-N,N,N′,N′- |
| | tetramethyluronium hexafluorophosphate; and |
| EtOH | ethanol; and |
| EtOAc | ethyl acetate. |

### EXAMPLE 1

This Example illustrates the preparation of N-[1-(3-phenyl-3-[cyclopenytlacetylamino]propyl)-4-piperidinyl]-N-ethyl-4-methanesulfonylphenylacetamide (Compound No. 1 of Table I).

A solution of cyclopentylacetic acid (0.005 mmol) in NMP (50 μL) was added to a solution of HATU (0.01 mmol) and diisopropylethylamine (0.03 mmol) in NMP (100 μL). To the resulting mixture was added N-[1-(3-phenyl-3-aminopropyl)-4-piperidinyl]-N-ethyl4-methanesulfonylphenylacetamide dihydrochloride (Method A) (0.005 mmol) in NMP (100 μL). The mixture was left at room temperature for 18 h, then evaporated. The residue was partitioned between DCM (250 μL) and water (250 μL) and the phases separated. The organic phase was concentrated giving the title compound which was characterised by LC-MS; MS: 568.

### EXAMPLE 2

This Example ilustrates the preparation of N-[1-(3-Phenyl-3-cyclobutylcarbonylaminopropyl)-4-piperidinyl]-N-allyl-4-methanesulfonylphenylacetamide (Compound No. 27 of Table I).

To a solution of N-[1-(3-phenyl-3-aminopropyl)-4-piperidinyl]-N-allyl-4-methanesulfonylphenylacetamide (60 mg, 0.13 mmol) in dichloromethane (DCM) (1 mL) was added diisopropylethylamine (130 μL, 0.75 mmol) and cyclobutane carboxylic acid (15 μL, 0.16 mmol) followed by HATU (100 mg, 0.26 mmol). The resulting mixture was stirred at room temperature for 18 h. The mixture was partitioned between water and DCM, the organic phase was washed with water and brine, dried (MgSO₄) and concentrated. The residue was purified by silica column chromatography (eluent 5% MeOH/DCM) to yield the title compound; NMR: 1.3 (m, 3H), 1.9 (m, 4H), 2.1 (m, 8H), 3.0 (m, 4H), 3.2 (s, 3H), 3.8 (s, 2H), 3.9 (s, 2H), 4.3 (m, 1H), 4.9 (m, 1H), 5.2 (m, 2H), 5.8 (m, 1H), 7.2 (m, 1H), 7.3 (m, 4H), 7.5 (d, 2H), 7.7 (d, 1H), 7.8 (d, 2H); MS: 552.

### EXAMPLE 3

This Example illustrates the preparation of N-[1-(3-Phenyl-3-cyclobutylcarbonylaminopropyl)-4-piperidinyl]-N-cyclopropyl-4-methanesulfonylphenylacetamide hydrochloride (Compound No. 28 of Table I).

To a stirred solution of hydroxybenztriazole (68 mg, 0.50 mmol) and diisopropylcarbodiimide (0.1 mL, 0.5 mmol) in DCM (3 mL) was added 4-methanesulfonylphenylacetic acid (109 mg, 0.5 mmol) and the resulting mixture stirred at room temperature for 1 h. A solution of 1-(3-phenyl-3-cyclobutylcarbonylaminopropyl)-4-cyclopropylaminepiperidine (90 mg, 0.25 mmol) in DCM (1 mL) was added and the resulting mixture stirred at room temperature for 20 h. The reaction mixture was eluted through an ISOLUTE™ SCX column with methanol followed by 2% aqueous ammonia/MeOH. The product was dissolved in DCM (5 mL) and ethereal HCl was added to give, after evaporation, the title compound (150 mg); NMR: 0.9 (m, 4H), 2.0 (m, 16H), 2.5 (m, 3H), 3.0 (m, 4H), 3.2 (s, 3H), 4.0 (s, 1H), 4.8 (m, 1H), 7.2 (m, 5H), 7.5 (d, 2H), 7.8 (d, 2H), 8.1 (d, 1H); MS: 552.

### EXAMPLE 4

This Example illustrates the preparation of (S)-N-[1-(3-phenyl-3-[benzocyclobutenyl-carboxyaminolpropyl)-4-piperidinyl]-N-ethyl-4-methanesulfonylphenylacetamide (Compound No. 1 of Table II).

To a mixture of (S)-N-[1-(3-phenyl-3-aminopropyl)-4-piperidinyl]-N-ethyl-4-methanesulfonylphenylacetamide dihydrochloride (Method S; 220 mg, 0.42 mmol) and DIPEA (0.75 mL) in DCM (5 mL) was added 1-benzocyclobutenecarboxylic acid (100 mg, 0.68 mmol). To the resulting mixture was added HATU (300 mg). The mixture was left at room temperature for 18 h, washed with 2M aqueous sodium hydroxide and water, then evaporated. Purification was achieved by BondElut chromatography eluting with a solvent mixture of ethyl acetate to 20% methanol in ethyl acetate to give the title compound; MS: 588.

The procedure described in Example 4 can be repeated using different carboxylic acids (such as indane-2-carboxylic acid and tetrahydropyran-4-carboxylic acid) in place of 1-benzocyclobutenecarboxylic acid or different amines (such as N-[1-(3-cyclohexyl-3-aminopropyl)4-piperidinyl]-N-ethyl-4-methanesulfonylphenylacetamide (Method V) or N-[1-(3-[4-chlorophenyl]-3-aminopropyl)-4-piperidinyl]-N-ethyl-4-methanesulfonylphenylacetamide (Method AA)) in place of (S)-N-[1-(3-phenyl-3-aminopropyl)4-piperidinyl)-N-ethyl-4-methanesulfonylphenylacetamide dihydrochloride.

Starting materials are commercially available, have been described in the literature or can be prepared by adaptation of literature methods. Examples of literature methods include: P. Richter, Ch. Garbe and G. Wagner, E. Ger. Pharmazie, 1974, 29(4), 256-262; C. Oniscu, D. Nicoara and G. Funieru, “4-(Ureidosulfonyl)phenylacetic acid and its ureide”, RO79-966646, (Romanian document); and M. A. Zahran, M. M. Ali, Y. A. Mohammed and A. A. Shehata, Int. J. Chem., 1993, 4(3), 61.

Method A

### Preparation of N-[1-(3-phenyl-3-aminopropyl)-4-piperidinyl]-N-ethyl-4-methanesulfonylphenylacetamide dihydrochloride

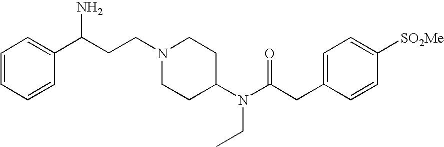

To a solution of 3-phenyl-3-Boc-aminopropanal (513 mg, 2.0 mmol) and N-(4-piperidinyl)-N-ethyl-4-methanesulfonylphenylacetamide (645 mg, 2.0 mmol) in methanol (15 mL) was added acetic acid (0.2 mL) and the resulting mixture was stirred at room temperature for 1 h. Sodium triacetoxyborohydride (844 mg, 4.0 mmol) was added and the mixture was stirred at room temperature for 18 h then evaporated. The residue was partitioned between DCM and water, and the organic phase was washed with brine, dried and concentrated. The residue was suspended in 4M HCl in dioxane (20 mL) and methanol (5 mL) was added. The resulting mixture was heated to reflux for 7 h, then cooled to room temperature and concentrated giving an oily residue which was purified by silica gel chromatography (eluent 5% MeOH/DCM then 10% MeOH/DCM) yielding the title compound as a solid (675 mg); NMR (d6 DMSO at 373K): 1.1 (t, 3H), 1.5 (m, 2H), 1.9 (m, 2H), 2.0 (m, 1H), 2.3 (m, 2H), 3.0 (m, 1H), 3.2 (m, 4H), 3.3 (q, 2H), 3.9 (s, 2H), 4.0 (m, 1H), 4.4 (m, 1H), 7.4 (m, 3H), 7.5 (m, 4H), 7.9 (m, 2H); MS: 458.

Method B

### Preparation of N-(4-piperidinyl)-N-ethyl-4-methanesulfonylphenylacetamide

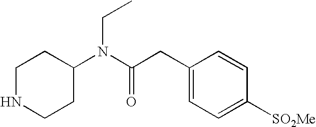

To a solution of N-(1-phenylmethyl-4-piperidinyl)-N-ethyl-4-methanesulfonylphenyl-acetamide (34 g, 82 mmol) in ethanol (600 mL) was added ammonium formate (40 g). The mixture was purged with argon and 30% Pd on carbon (4.2 g) was added. The resulting mixture was stirred at reflux for 4 h, then allowed to cool and filtered through diatomaceous earth. The filtrate was evaporated to give a thick oil which solidified on standing to yield the title compound (24.9 g. 77 mmol); NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H), 3.80 and 3.87 (s, 2H), 7.50 (m, 2H), 7.85 (m, 2H); MS: 325.

Method C

### Preparation of N-(1-phenylmethyl-4-piperidinyl)-N-ethyl-4-methanesulfonylphenylacetamide

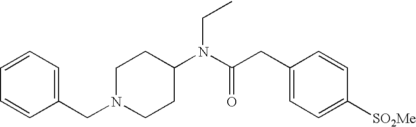

To a solution of 1-phenylmethyl-4-ethylaminopiperidine dihydrochloride (32.0 g, 110 mmol) in DCM (500 mL) was added N,N-diisopropylethylamine (60 mL) with stirring to ensure complete dissolution. 4-Methanesulfonylphenylacetic acid (25.0 g, 117 mmol), 4-dimethylaminopyridine (4-DMAP) (2.0 g) and dicyclohexylcarbodiimide (DCCI) (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent 10% MeOH/ethyl acetate) to afford the title compound (35 g, 76%); NMR: 1.00 and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.45 (s, 2H), 3.80 and 3.87 (s, 2H), 3.70 and 4.10 (m, 1H), 7.2-7.3 (m, 5H), 7.48 (m, 2H), 7.82 (m, 2H); MS: 415.

Method D

### Preparation of 1-phenylmethyl-4-ethylaminopiperidine dihydrochloride

To a solution of 1-phenylmethyl-4-piperidone (25.0 g, 132 mmol) in THF (250 mL) was added ethylamine hydrochloride (12.0 g, 147 mmol) and methanol (50 mL) and the resulting mixture stirred at room temperature for 10 min. Sodium triacetoxyborohydride (40 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4-ethylaminopiperidine as an oil. This was dissolved in ethanol (50 mL) and concentrated hydrochloric acid (20 mL) was added. The resulting crystals were collected, washed with diethyl ether and dried giving the title compound as a solid (38 g); NMR (CDCl3): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H); MS: 219.

Method E

### Preparation of 3-phenyl-3-Boc-aminopropanal

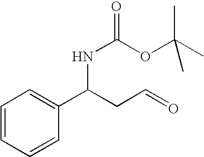

A solution of 3-phenyl-2-Boc-aminopropanol (700 mg, 2.78 mmol) in DCM (8 mL) was added to a stirred solution of (1,1,1-triacetoxy)-1,1-dihydro-1,2-benziodoxol-3(1H)-one (1.30 g, 3.06 mmol) in DCM (5 mL) at room temperature followed by pyridine (0.3 mL). After stirring for 6 h at room temperature the mixture was partitioned between diethyl ether and saturated aqueous sodium bicarbonate solution containing sodium thiosulfate. The organic phase was washed with water and brine, dried and concentrated giving the title compound as a solid (790 mg); NMR: 1.4 (s, 9H), 2.8 (m, 2H), 5.1 (m, 1H), 7.3 (m, 5H), 8.6 (m, 1H), 9.6 (t, 1H).

Method F

### Preparation of 3-phenyl-3-Boc-aminopropanol

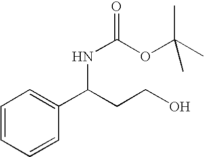

To a solution of 3-phenyl-3-Boc-aminopropanoic acid (1.0 g, 3.78 mmol) in THF (10 mL) was added borane-THF complex (7.5 mL, 1.5M, 11.3 mmol) at 0° C. The resulting mixture was stirred with warming to room temperature for 5 h. 10% Acetic acid in methanol (20 mL) was added dropwise, the resulting mixture was concentrated and the residue partitioned between DCM and 1M aqueous HCl. The organic phase was washed with water and brine, dried (MgSO₄) and concentrated. The residue was purified by Bond Elut chromatography (eluent 5% MeOH/DCM) to afford the title compound (900 mg).

Method G

### Preparation of 3-phenyl-3-Boc-aminopropanoic acid

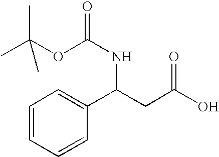

To a solution of DL-3-amino-3-phenylpropanoic acid (5 g, 30.2 mmol) in 2M aqueous sodium hydroxide (70 mL) was added a solution of di-tert-butyldicarbonate (8.56 g, 39.2 mmol) in THF (60 mL) and the resulting mixture stirred at room temperature for 48 h. Water (50 mL) was added and the mixture washed twice with ethyl acetate (50 mL). The aqueous phase was acidified to pH 3 with concentrated aqueous HCl, and the resulting mixture was extracted twice with ethyl acetate (60 mL). The combined organic extracts were dried (MgSO₄) and concentrated to give the title compound as a white solid (4.8 g); NMR: 1.4 (s, 9H), 2.7 (m, 2H), 4.8 (m, 1H), 7.3 (m, 5H), 7.5 (br d, 1H), 12.1 (br s, 1H); MS: 266.

Method H

### Preparation of N-[1-(3-phenyl-3-aminopropyl)-4-piperidinyl]-N-allyl-4-methanesulfonylphenylacetamide

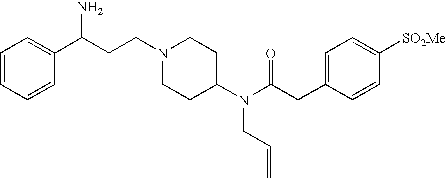

To a solution of 3-phenyl-3-Boc-aminopropanal (513 mg, 2.0 mmol) and N-(4-piperidinyl)-N-allyl-4-methanesulfonylphenylacetamide (500 mg, 1.48 mmol) in methanol (10 mL) was added acetic acid (0.5 mL) and the resulting mixture was stirred at room temperature for 1 h. Sodium triacetoxyborohydride (593 mg, 2.8 mmol) was added and the mixture was stirred at room temperature for 18 h then evaporated. The residue was partitioned between DCM and water, and the organic phase was washed with brine, dried and concentrated. The residue was suspended in 4M HCl in dioxane (20 mL) and methanol (5 mL) was added. The resulting mixture was heated to reflux for 7 h, then cooled to room temperature and concentrated giving an oily residue which was purified by silica gel chromatography (eluent 5% 2M NH₃/MeOH/DCM then 10% 2M NH₃/MeOH/DCM) yielding the title compound (60 mg); MS: 470.

Method I

### Preparation of N-(4-piperidinyl)-N-allyl-4-methanesulfonylphenylacetamide

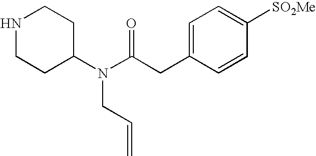

A solution of N-(1-phenylmethyl-4-piperidinyl)-N-allyl-4-methanesulfonylphenylacetamide (4.40 g, 10.3 mmol) in DCM (30 mL) was cooled in an ice-water bath under an argon atmosphere. 1-Chloroethyl chloroformate (1.34 mL, 12.4 mmol) was added and the resulting mixture was stirred for 3 h while warming to room temperature. The mixture was evaporated and the residue dissolved in methanol (30 mL). The resulting mixture was refluxed for 1 h, allowed to cool and concentrated. The crude product was purified by silica column chromatography (eluent 5% EtOH/DCM then 15% EtOH/2% isopropylamine/DCM) to give the title compound (1.30 g); NMR: 1.50 (m, 4H), 2.50 (m, 2H), 2.95 (m, 2H), 3.20 (s, 3H), 3.74 and 3.91 (s, 1H), 3.80 and 3.95 (d, 1H), 4.29 (m, 1H), 5.00 and 5.05 (d, 1H), 5.20 (m, 1H), 5.73 and 5.89 (dddd, 1H), 7.44 and 7.49 (d, 2H), 7.85 (m, 2H).

Method J

### Preparation of 1-(3-phenyl-3-cyclobutylcarbonylaminopropyl)-4-cyclopropylaminopiperidine

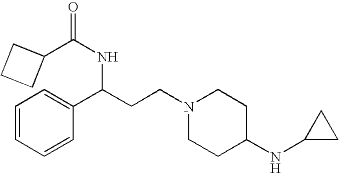

To a solution of 1-(3-phenyl-3-cyclobutylcarbonylaminopropyl)-4-piperidone (150 mg, 0.48 mmol) in 10% acetic acid/DCM (6 mL) was added cyclopropylamine (36 μL, 0.53 mmol) and the resulting mixture was stirred at room temperature for 30 min. Sodium triacetoxyborohydride (163 mg, 0.77 mmol) was added and the mixture stirred for a further 20 h. The reaction was quenched by the addition of saturated aqueous sodium bicarbonate solution (15 mL) and the resulting mixture extracted three times with DCM. The combined organic extracts were dried (MgSO₄) and concentrated giving an oil which was purified by silica column chromatography (eluent 5% ethanol/DCM then 1% isopropanol/10% ethanol/DCM) affording the title compound (100 mg); MS: 356.

Method K

### Preparation of 1-(3-phenyl-3-cyclobutylcarbonylaminopropyl)-4-piperidone

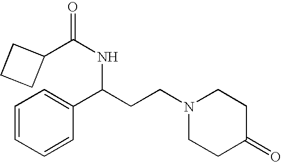

To a solution of cyclobutane carboxylic acid (0.59 mL, 6.2 mmol) in DCM (15 mL) was added a few drops of DMF followed by oxalyl chloride (0.54 mL, 6.2 mmol). The resulting mixture was stirred at room temperature for 1 h. The mixture was then added to a solution of 1-(3-phenyl-3-aminopropyl)-4-piperidone (480 mg, 2.1 mmol) and triethylamine (0.58 mL, 4.1 mmol) in DCM (15 mL) and the resulting mixture was stirred at room temperature for 20 h. The mixture was partitioned between aqueous potassium carbonate solution and DCM. The organic phase was dried (MgSO₄) and concentrated and the crude product was purified by silica column chromatography (eluent 5% EtOH/DCM then 10% EtOH/DCM) affording the title compound (150 mg); MS: 315.

Method L

### Preparation of 1-(3-phenyl-3-aminopropyl)-4-piperidone

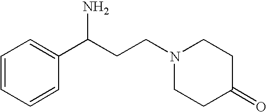

1-(3-Phenyl-3-Boc-aminopropyl)-4-piperidone ethylene ketal (2.13 g, 5.66 mmol) was mixed with 6M HCl (50 mL) and the mixture was heated to reflux for 2 h. After cooling to room temperature the mixture was made basic with sodium hydroxide solution and extracted three times with DCM. The combined organic extracts were washed with brine, dried (MgSO₄) and concentrated. The crude product was purified by silica column chromatography (eluent 4% EtOH/DCM then 1% ammonia/5% EtOH/DCM) affording the title compound (490 mg); NMR (CDCl3): 1.7 (m, 2H), 2.0 (m, 2H), 2.4 (m, 8H), 4.6 (m, 1H), 7.2 (m, 7H); MS: 233.

Method M

### Preparation of 1-(3-phenyl-3-Boc-aminopropyl)-4-piperidone ethylene ketal

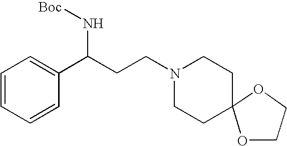

3-Phenyl-3-Boc-aminopropanal (4.14 g, 16.6 mmol) and 1,4-dioxa-8-azaspiro(4,5)decane (2.14 mL, 16.6 mmol) were dissolved in 10% acetic acid/DCM (180 mL) and the resulting mixture stirred at room temperature for 10 min. Sodium triacetoxyborohydride (5.29 g, 24.9 mmol) was added and the mixture stirred for a further 2 h. The reaction was quenched by the addition of saturated aqueous sodium bicarbonate solution and the resulting mixture extracted three times with DCM. The combined organic extracts were dried (MgSO₄) and concentrated. The residue was purified by silica column chromatography (eluent 4% ethanoVDCM) affording the title compound as an oil (2.14 g); NMR: 1.3 (s, 9H), 1.6 (t, 4H), 1.8 (m, 2H), 2.25 (t, 2H), 2.4 (m, 5H), 3.8 (s, 4H), 7.25 (m, 5H); MS: 377.

Method N

### Preparation of N-(4-piperidinyl)-N-cyclopropyl-4-methanesulfonylphenylacetamide

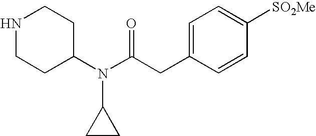

This was prepared by the reaction of N-(1-phenylmethyl-4-piperidinyl)-N-cyclopropyl-4-methanesulfonylphenylacetamide according to the procedure used for Method B; NMR: 0.7-0.9 (m, 4H), 1.5 (d, 2H), 1.8 (m, 2H), 2.2 (dd, 2H), 2.6 (m, 1H), 2.9 (d, 2H), 3.15 (s, 3H), 3.85 (m, 1H), 3.9 (s, 2H), 7.45 (d, 2H), 7.8 (d, 2H); MS: 337.

Method O

### Preparation of N-(1-phenylmethyl-4-piperidinyl)-N-cyclopropyl-4-methanesulfonylphenylacetamide

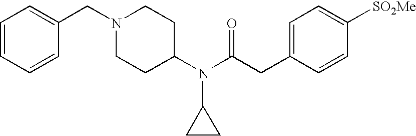

This was prepared by the reaction of 1-phenylmethyl-4-cyclopropylaminopiperidine with 4-methanesulfonylphenylacetic acid according to the procedure used for Method C; NMR: 0.7-0.9 (m, 4H), 1.55 (d, 2H), 1.9 (m, 4H), 2.6 (m, 1H), 2.8 (d, 2H), 3.15 (s, 3H), 3.4 (s, 2H), 3.8 (m, 1H), 3.95 (s, 2H), 7.1-7.3 (m, 5H), 7.45 (d, 2H), 7.8 (d, 2H); MS: 427;

Method P

### Preparation of 1-phenylmethyl-4-cyclopropylaminopiperidine

This was prepared by reacting 1-phenylmethyl-4-piperidone with cylopropylamine according to the procedure used for Method D; NMR: 0.0 (m, 2H), 0.2 (m, 2H), 1.1 (m, 2H), 1.65 (d, 2H), 1.75-2.0 (m, 4H), 2.3 (m, 1H), 2.6 (m, 1H), 3.3 (s, 2H), 7.0-7.2 (m, 5H); MS: 231.

Method Q

### Preparation of N-(1-phenylmethyl-4-piperidinyl)-N-allyl-4-methanesulfonylphenylacetamide

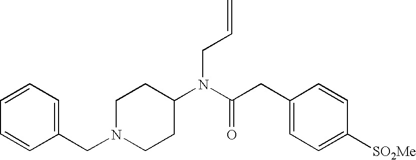

This was prepared by reacting 1-phenylmethyl-4-allylamine with 4-methanesulfonylphenylacetamide according to the procedure used for Method C; NMR (d6-DMSO, 373K): 1.65 (m, 2H), 1.88 (m, 2H), 2.39 (m, 2H), 3.05 (m, 2H), 3.09 (s, 3H), 3.75 (m, 4H), 3.93 (s, 2H), 4.08.(m, 1H), 5.15 (m, 2H), 5.82 (dddd, 1H), 7.30 (m, 5H), 7.45 (d, 2H), 7.80 (d, 2H).

Method R

### Preparation of 1-phenylmethyl-4-allylamine

This was prepared by reacting 1-phenylmethyl-4-piperidone with allylamine according to the procedure used for Method D; NMR (CDCl3): 1.4 (m, 2H), 1.5 (m, 2H), 1.9 (m, 2H), 2.0 (dd, 2H), 2.5 (m, 1H), 2.8 (m, 2H), 3.3 (d, 2H), 3.5 (s, 3H), 5.1 (d, 1H), 5.9 (dddd, 1H), 7.3 (m, 5H); MS: 231.

Method S

### (S)-N-[1-(3-Phenyl-3-aminopropyl)-4-piperidinyl]-N-ethyl-4-methanesulfonylphenylacetamide dihydrochloride

### Step 1: Preparation of 1-phenylmethyl-4-ethylaminopiperidine dihydrochloride

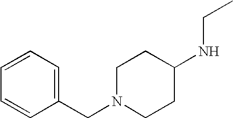

To a solution of 1-phenylmethyl-4-piperidone (25.0 g, 132 mmol) in THF (250 mL) was added ethylamine hydrochloride (12.0 g, 147 mmol) and methanol (50 mL) and the resulting mixture stirred at room temperature for 10 min. Sodium triacetoxyborohydride (40 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4-ethylaminopiperidine as an oil. This was dissolved in ethanol (500 mL) and concentrated hydrochloric acid (20 mL) was added. The resulting crystals were collected, washed with diethyl ether and dried giving the sub-titled compound as a solid (38 g); NMR:(CDCl₃): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H); MS: 219 (MH+).

### Step 2: Preparation of N-(1-Phenylmethyl-4-piperidinyl)-N-ethyl-4-methanesulfonylphenylacetamide

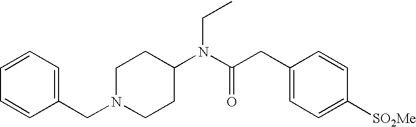

To a solution of 1-phenylmethyl-4-ethylaminopiperidine dihydrochloride (32.0 g, 110 mmol) in DCM (500 mL) was added N,N-diisopropylethylamine (60 mL) with stirring to ensure complete dissolution. 4-Methanesulfonylphenylacetic acid (25.0 g, 117 mmol), 4-Dimethylaminopyridine (4-DMAP) (2.0 g) and dicyclohexylcarbodiimide (DCCI) (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent 10% MeOH/ethyl acetate) to afford the sub-titled compound (35 g, 76%); NMR: 1.00 and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.45 (s, 2H), 3.80 and 3.87 (s, 2H), 3.70 and 4.10 (m, 1H), 7.2-7.3 (m, 5H), 7.48 (m, 2H), 7.82 (m, 2H); MS: 415 (MH+).

### Step 3: Preparation of N-(4-Piperidinyl)-N-ethyl-4-methanesulfonylphenylacetamide

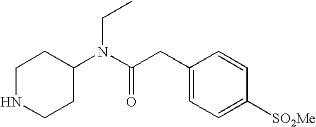

To a solution of N-(1-phenylmethyl-4-piperidinyl)-N-ethyl-4-methanesulfonylphenyl-acetamide (34 g, 82 mmol) in ethanol (600 mL) was added ammonium formate (40 g). The mixture was purged with argon and 30% Pd on carbon (4.2 g) was added. The resulting mixture was stirred at reflux for 4 h, then allowed to cool and filtered through diatomaceous earth. The filtrate was evaporated to give a thick oil which solidified on standing to yield the sub-titled compound (24.9 g, 94%); NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H), 3.80 and 3.87 (s, 2H), 7.50 (m, 2H), 7.85 (m, 2H); MS: 325 (MH+).

### Step 4: Preparation of Title Compound

To a solution of (S)-3-phenyl-3-Bocaminopropanal (Method B, 1.4 g, 5.6 mmol) in ethanol (100 mL) and DCM (50 mL) was added N-(4-piperidinyl)-N-ethyl-4-methanesulfonylphenylacetamide (2.0 g, 6.2 mmol), glacial acetic acid (0.6 mL, 10 mmol) and sodium triacetoxyborohydride (2.0 g, 9.4 mmol) and the resulting mixture was stirred at room temperature for 18 h. The mixture was partitioned between DCM and 2M aqueous sodium hydroxide (35 mL), and the organic phase was washed with water, dried and concentrated. The residue was suspended in methanol (10 mL) and concentrated hydrochloric acid (10 mL) was added. The resulting mixture was stirred for 30 min. then evaporated. The residue was azeotroped with ethanol and toluene and triturated with diethyl ether yielding the title compound as a solid (1.3 g); NMR (d6 DMSO at 373K): 1.1 (t, 3H), 1.5 (m, 2H), 1.9 (m, 2H), 2.0 (m, 1H), 2.3 (m, 2H), 3.0 (m, 1H), 3.2 (m, 4H), 3.3 (q, 2H), 3.9 (s, 2H), 4.0 (m, 1H), 4.4 (m, 1H), 7.4 (m, 3H), 7.5 (m, 4H), 7.9 (m, 2H); MS: 458.

Method T

### (S)-3-Phenyl-3-Boc-aminopropanal

To a solution of (S)-N-methyl-N-methoxy-3-phenyl-3-Bocaminopropionamide (Method U, 5.52 g, 17.9 mmol) in toluene (180 mL) at −20° C. was added sodium bis(2-methoxyethoxy)aluminium hydride (65% solution in toluene, 35.8 mmol) dropwise. The resulting mixture was stirred at −15° C. for 1 h. The mixture was washed with saturated aqueous sodium dihydrogen phosphate solution (250 mL). The organic phase was dried (Na₂SO₄) and concentrated to give the title compound (5 g); NMR: 1.4 (s, 9H), 2.8 (m, 2H), 5.1 (m, 1H), 7.3 (m, 5H), 8.6 (m, 1H), 9.6 (t, 1H).

Method U

### (S)-N-Methyl-N-methoxy-3-phenyl-3-Bocaminopropionamide

To a solution of (S)-3-phenyl-3-Bocaminopropanoic acid (available from PepTech Corp. of Cambridge, Mass., USA; 4.97 g, 18.7 mmol) in DCM (100 mL) was added DIPEA (14.8 mL, 84.8 mmol) and N,O-dimethylhydroxylamine hydrochloride (2.21 g, 22.7 mmol) followed by HATU (8.44 g, 84.8 mmol). The resulting mixture was stirred at room temperature for 18 h, diluted with DCM, washed with 2M aqueous sodium hydroxide and water. The organic phase was dried (Na₂SO₄) and concentrated. The residue was purified by silica column chromatography (eluting with isohexane then 3:1 ethyl acetate to isohexane) giving the title compound as a colourless oil (5.58 g, 97%); NMR (CDCl₃): 1.40 (s, 9H), 2.83 (dd, 1H), 3.01 (m, 1H), 3.08 (s, 3H), 3.52 (s, 3H), 5.10 (m, 1H), 7.28 (m, 5H); MS: 309.

Method V

### N-[1-(3-Cyclohexyl-3-aminopropyl)-4-piperidinyl]-N-ethyl-4-methanesulfonylphenylacetamide

N-[1-(3-Cyclohexyl-3-Bocaminopropyl)-4-piperidinyl]-N-ethyl-4-methanesulfonylphenylacetamide (Method W, 9.4 g, 20 mmol) was dissolved in trifluoroacetic acid (30 mL) and the resulting mixture was stirred at room temperature for 2 h. Evaporation gave the title compound (3.6 g); NMR: 0.8-1.85 (m, 25H), 2.3 (m, 3H), 2.8 (m, 2H), 3.1 (s, 3H+H₂O), 3.8 (d, 2H), 7.4 (d, 2H), 7.75 (m, 2H).

Method W

### N-[1-(3-Cyclohexyl-3-Bocaminopropyl)-4-piperidinyl]-N-ethyl-4-methanesulfonylphenylacetamide

To a mixture of 3-cyclohexyl-3-Boc-aminopropanal (Method X, 7 g, 27 mmol) and N-(4-piperidinyl)-N-ethyl-4-methanesulfonylphenylacetamide (9.6 g, 27 mmol) in DCM (200 mL) and ethanol (20 mL) was added acetic acid (0.5 mL). The resulting mixture was stirred at room temperature for 30 min. before the addition of sodium triacetoxyborohydride (5.8 g, 27 mmol). The resulting mixture was stirred at room temperature for 18 h. The reaction mixture was washed with 2M aqueous sodium hydroxide (3×50 mL), dried and evaporated. The residue was purified by silica gel chromatography (eluent DCM then ethyl acetate then 10% methanol in ethyl acetate) giving the title compound (9.4 g); NMR: 0.8-1.1 (m, 5H), 1.18 (s, 9H), 1.2-2 (m, 11H), 2.2 (m, 2H), 2.8 (m, 2H), 3.3 (s, 3H), 3.8 (d, 2H), 6.5 (d, 1H), 7.5 (m, 2H), 7.8 (m, 2H).

Method X

### 3-Cyclohexyl-3-Boc-aminopropanal

To a solution of N-methyl-N-methoxy-3-cyclohexyl-3-Bocaminopropionamide (Method Y, 9.9 g, 31 mmol) in toluene (100 mL) at 0° C. was added sodium bis(2-methoxyethoxy)aluminium hydride (65% solution in toluene, 31 mmol) dropwise. The resulting mixture was stirred at 0° C. for 2 h. 2M aqueous sodium hydroxide was added and the mixture warmed to room temperature and filtered. The filtrate was washed with 2M aqueous sodium hydroxide (2×20 mL), dried and evaporated giving the title compound (7 g) which was used in the next reaction without characterisation.

Method Y

### N-Methyl-N-methoxy-3-cyclohexyl-3-Bocaminopropionamide

To a solution of 3-cyclohexyl-3-Bocaminopropionic acid (Method Z, 8.6 g, 32 mmol) and HBTU (12.3 g, 32 mmol) in DMF was added triethylamine (32 mmol) and the resulting mixture was stirred at room temperature for 10 min. N,O-dimethylhydroxylamine hydrochloride (3.3 g, 32 mmol) was added and the resulting mixture was stirred at room temperature for 18 h before being evaporated. The residue was dissolved in ethyl acetate and the solution washed with water (3×75 mL), dried and evaporated giving the title compound (9.9 g); NMR: 0.8-1.2 (m, 6H), 1.6 (m, 5H), 2.4 (m, 1H), 3 (s, 3H), 3.05 (m, 1H), 3.6 (s, 3H), 3.7 (m, 1H), 6.5 (d, 1H).

Method Z

### 3-Cyclohexyl-3-Bocaminopropionic acid

To a mixture of 3-cyclohexyl-3-aminopropionic acid (5 g, 30 mmol), TBF (20 mL) and 2M aqueous sodium hydroxide (30 mL, 58 mmol) was added di-tert-butyldicarbonate (9.3 g, 43 mmol) and the resulting mixture was stirred at room temperature for 8 h. Water (50 mL) was added and the mixture extracted with DCM (2×50 mL). The aqueous phase was acidified to pH 2 and extracted with DCM (5×25 mL). The combined organic extracts were dried and evaporated giving the title compound (8.6 g); NMR: 0.8-1.8 (m, 11H), 2.1-2.4 (m, 2H), 3.6 (m, 1H), 6.6 (d, 1H), 11.95 (s, 1H).

Method AA

### N-[1-(3-[4-chlorophenyl]-3-aminopropyl)-4piperidinyl]-N-ethyl-4-methanesulfonylphenylacetamide

This was prepared from 3-(4-chlorophenyl)-3-aminopropanoic acid using a similar sequence of reactions to that used to prepare N-[1-(3-cyclohexyl-3-aminopropyl)-4-piperidinyl]-N-ethyl-4-methanesulfonylphenylacetamide from 3-cyclohexyl-3-aminopropionic acid (Methods V-Z).

### EXAMPLE 5

The ability of compounds to inhibit the binding of RANTES or MIP-1α (was assessed by an in vitro radioligand binding assay. Membranes were prepared from Chinese hamster ovary cells which expressed the recombinant human CCR5 receptor. These membranes were incubated with 0.1 nM iodinated RANTES or MIP-1α, scintillation proximity beads and various concentrations of the compounds of the invention in 96-well plates. The amount of iodinated RANTES or MIP-1α bound to the receptor was determined by scintillation counting. Competition curves were obtained for compounds and the concentration of compound which displaced 50% of bound iodinated RANTES or MIP-1α was calculated (IC₅₀). Certain compounds of formula (I) had an IC₅₀ of less than 50 μM.

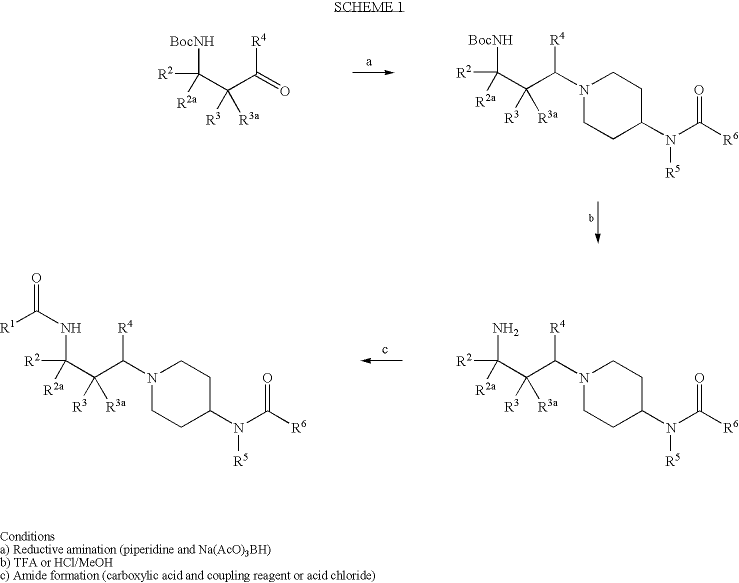

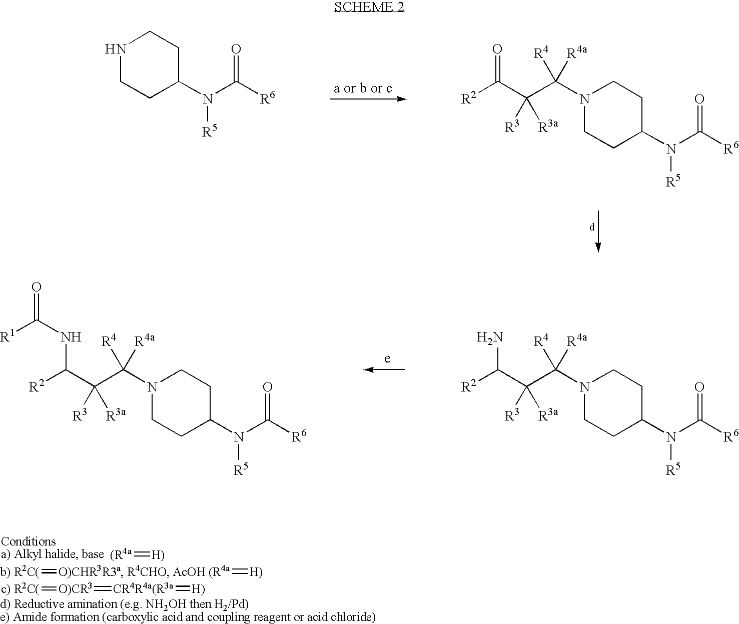

1. A compound of formula (I): 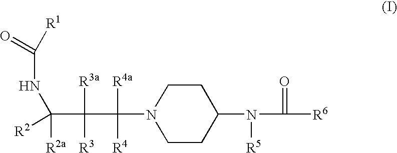
wherein:
R¹ is C₃₋₇ cycloalkyl, C₄₋₇ cycloalkyl fused to a phenyl ring, C₅₋₇ cycloalkenyl, heterocyclyl (itself optionally substituted by oxo or C₁₋₄ alkyl), C₁₋₈ alkyl (substituted by C₃₋₆ cycloalkyl, C₅₋₆ cycloalkenyl, S(O)ₚR⁷ or COR⁸), C₂₋₈ alkenyl or C₂₋₈ alkynyl;
R² is optionally substituted phenyl, optionally substituted heteroaryl or cycloalkyl;
R^{2a}, R⁴ and R^{4a} are, independently, hydrogen or C₁₋₄ alkyl;
R³ and R^{3a} are, independently, hydrogen or C₁₋₄ alkyl or C₁₋₄ alkoxy;
R⁵ is hydrogen, C₁₋₄ alkyl (optionally substituted by halogen, hydroxy, C₁₋₄ alkoxy, C₃₋₇ cycloalkyl, SH, C₁₋₄ alkylthio, cyano or S(O)_{q}(C₁₋₄ alkyl)), C₃₋₄ alkenyl, C₃₋₄ alkynyl or C₃₋₇ cycloalkyl;
R⁶ is phenyl, heteroaryl, phenylNH, heteroarylNH, phenyl(C₁₋₂)alkyl, heteroaryl(C₁₋₂ )alkyl, phenyl(C₁₋₂ alkyl)NH or heteroaryl(C₁₋₂ alkyl)NH;
R⁷ and R⁸ are, independently, C₁₋₄ alkyl;
wherein the phenyl and heteroaryl rings of any of the foregoing are independently optionally substituted by halo, cyano, nitro, hydroxy, C₁₋₄ alkyl, C₁₋₄ alkoxy, S(O)ₘC₁₋₄ alkyl, S(O)₂NR⁹R¹⁰, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃;
R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl);
m, p and q are, independently, 0, 1 or 2;
provided that when heterocyclyl contains a one heteroatom and that heteroatom is nitrogen, then the heterocyclyl ring is not N-linked to the remainder of the structure of formula (I); and provided that when R¹ is cyclobutyl or tetrahydropyran, R² is optionally substituted phenyl, R³ is hydrogen or alkoxy and R⁶ is benzyl (optionally substituted by alkoxy) or pyridinylmethyl, then R^{2a}, R^{3a}, R⁴, R^{4a} and R⁵ are not all hydrogen;
or a pharmaceutically acceptable salt thereof or a solvate thereof.

2. A compound as claimed in claim 1 wherein R¹ is cyclopropyl, cyclobutyl, cyclopentyl, cyclohexenyl, benzocyclobuten-1-yl, indanyl, 5-, 6- or 8-membered, non-N-linked, heterocyclyl (optionally substituted by oxo or methyl), C₁₋₄ alkyl (singly substituted by C₃-6 cycloalkyl, C₅₋₆ cycloalkenyl, S(C₁₋₄ alkyl) or CO(C₁₋₄ alkyl)), C₂₋₆ alkenyl or C₂₋₆ alkynyl.

3. A compound as claimed in claim 1 or 2 wherein R² is phenyl optionally substituted by halogen or CF₃.

4. A compound as claimed in claim 1, 2 or 3 wherein R^{2a} is hydrogen.

5. A compound as claimed in claim 1, 2, 3 or 4 wherein R³ and R^{3a} are both hydrogen.

6. A compound as claimed in claim 1, 2, 3, 4 or 5 wherein R⁴ in hydrogen or methyl and R^{4a} is hydrogen.

7. A compound as claimed in any one of the foregoing claims wherein R⁵ is ethyl, allyl or cyclopropyl.

8. A compound as claimed in any one of the foregoing claims wherein R⁶ is benzyl optionally substituted by S(O)₂(C₁₋₄)alkyl or S(O)₂NR⁹R¹⁰; wherein R⁹ and R¹⁰ are, independently, hydrogen or C₁₋₄ alkyl, or together with a nitrogen or oxygen atom, may join to form a 5- or 6-membered ring which is optionally substituted with C₁₋₄ alkyl, C(O)H or C(O)(C₁₋₄ alkyl).

9. A process for the preparation of a compound of formula (I) as claimed in claim 1 to 8 comprising treating a compound of formula (II): 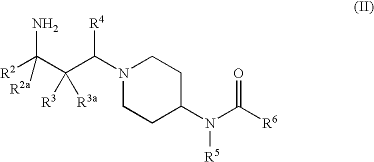
with:
an acid chloride of formula R¹ C(O)Cl, in the presence of a base and in a suitable solvent; or,
an acid of formula R¹ CO₂H, in the presence of a suitable coupling agent, a suitable base and in a suitable solvent.

10. A pharmaceutical composition which comprises a compound of the formula (I), or a pharmaceutically acceptable salt thereof or solvate thereof as claimed in claim 1 to 8, and a pharmaceutically acceptable adjuvant, diluent or carrier.

11. A compound of the formula (I), or a pharmaceutically acceptable salt thereof or solvate thereof as claimed in claim 1 to 8, for use in therapy.

12. A compound of formula (I), or a pharmaceutically acceptable salt thereof or solvate thereof as claimed in claim 1 to 8, in the manufacture of a medicament for use in therapy.

13. A method of treating a chemokine mediated disease state in a warm blooded animal suffering from, or at risk of, said disease, which comprises administering to an animal in need of such treatment a therapeutically effective amount of a compound of formula (I), or a pharmaceutically acceptable salt thereof or solvate thereof as claimed in claim 1 to 8.
